(19) United States
(12) Patent Application Publication
(10) Pub. No.: US 2017/0143613 A1
(43) Pub. Date: May 25, 2017

(54) OIL-FREE EMOLLIENTS IN PERSONAL CARE COMPOSITIONS

(71) Applicant: Rohm and Haas Company, Philadelphia, PA (US)

(72) Inventors: Xiaodong Lu, North Wales, PA (US); Curtis Schwartz, Ambler, PA (US)

(21) Appl. No.: 15/318,450

(22) PCT Filed: Jun. 16, 2015
(86) PCT No.: PCT/US2015/035916

§ 371 Date: Dec. 13, 2016

Related U.S. Application Data
(60) Provisional application No. 62/015,872, filed on Jun. 23, 2014.

(51) Int. Cl. A61K 8/86; A61Q 19/00

(52) U.S. Cl. CPC A61K 8/86; A61Q 19/00; A61K 2800/30

(57) ABSTRACT

Provided are compositions that are useful as oil-free emollients in personal care formulations. The emollients are oil-soluble polyalkylene glycols, wherein the polyalkylene glycol comprises units derived from propylene oxide and units derived from butylene oxide, and in certain embodiments are compounds of Formula I:

RO-(PO)ₙ-(BO)ₘ-H (I)

wherein PO is propyleneoxy, BO is butyleneoxy, R is a linear or branched C₈-C₂₀ alkyl, n has an average value of from 5 to 20, and m has an average value of from 4 to 16.

### FIELD OF THE INVENTION

This invention relates generally to oil-free emollients that are useful in personal care formulations. The emollients are oil-soluble polyalkylene glycols.

### BACKGROUND

Personal care compositions are important products for most consumers. Personal care compositions contain a variety of additives that provide a wide array of benefits to the composition. Emollients are among the additives commonly used in personal care compositions to aid in moisturization of the skin, and in particular prevent and treat dry skin, protect sensitive skin, improve skin tone and texture, and mask imperfections. Emollients can increase skin hydration or act as a barrier to prevent trans-epidermal water loss (“TEWL”). It is also important for emollients to provide a desirable sensory feel to keep the skin in a smooth and supple condition, without suffering from negative aesthetic qualities, such as greasiness or stickiness.

Oil-free skin care products were originally developed to avoid using substances with high comedogenic potential, such as mineral oil, petrolatum, esters, and triglycerides. Many oil-free products in the skin care market do not contain hydrophobic ingredients, which may provide short term skin hydration, but lack the benefit of a barrier to TEWL. Emulsion-type oil free compositions have typically utilized silicone fluids, for example as disclosed in U.S. Pat. No. 5,380,528, although such compositions are generally considered to be sensory modifiers with poor performance as a barrier to TEWL. Another consideration surrounding the use of silicone fluids is the acknowledged need to replace such components due to environmental and health related issues.

Oil-free polyalkylene glycols have also been disclosed in personal care compositions. For example, U.S. Pat. No. 4,511,554 discloses an antiperspirant stick composition having a low staining potential, which contains a mixture of polyoxyethylene(25)propylene glycol stearate and a polyoxypropylene, polyoxyethylene ether of a long chain fatty alcohol. However, the prior art does not disclose a polyalkylene glycol according to the present invention which gives superior results as an oil-free skin care emollient.

Consequently, there is a need to develop new oil-free emollient compositions for use in personal care, including compositions that improve moisturization and hydration of skin while also providing desirable aesthetic and sensorial properties.

### STATEMENT OF INVENTION

One aspect of the invention provides a personal care composition comprising (a) an oil soluble polyalkylene glycol comprising units derived from propylene oxide and units derived from butylene oxide, and (b) a dermatologically acceptable carrier.

In another aspect, the invention provides a personal care composition comprising a dermatologically acceptable carrier and an oil-soluble polyalkylene glycol of Formula I:

RO-(PO)ₘ-(BO)ₘ-H (I)

wherein PO is propyleneoxy, BO is butyleneoxy, R is a linear or branched C₈-C₂₀ alkyl, n has an average value of from 5 to 20, and m has an average value of from 4 to 16.

In another aspect, the inventive personal care composition further comprises a personal care active.

Another aspect of the invention provides a method for treating skin comprising topically administering to the skin a composition comprising (a) an oil soluble polyalkylene glycol comprising units derived from propylene oxide and units derived from butylene oxide, and (b) a dermatologically acceptable carrier.

In yet another aspect, the invention provides a method for reducing trans-epidermal water loss in skin comprising topically administering to the skin a composition comprising (a) an oil soluble polyalkylene glycol comprising units derived from propylene oxide and units derived from butylene oxide, and (b) a dermatologically acceptable carrier.

### DETAILED DESCRIPTION

The inventors have now surprisingly found that oil-soluble polyalkylene glycols comprising units derived from propylene oxide and units derived from butylene oxide provide oil-like emolliency, sensorial feel, and moisturization in oil-free personal care formulations without exhibiting the high comedogenic potential that is typical of other conventional emollients. Accordingly, the present invention provides in one aspect a personal care composition comprising an oil-soluble polyalkylene glycol and a dermatologically acceptable carrier.

In the present invention, “personal care” is intended to refer to cosmetic and skin care compositions for leave on application to the skin, such as lotions, creams, gels, gel creams, serums, toners, wipes, liquid foundations, make-ups, tinted moisturizer, oils, face/body sprays, topical medicines, and sunscreens. “Personal care” relates to compositions to be topically administered (i.e., not ingested). Preferably, the personal care composition is cosmetically acceptable. “Cosmetically acceptable” refers to ingredients typically used in personal care compositions, and is intended to underscore that materials that are toxic when present in the amounts typically found in personal care compositions are not contemplated as part of the present invention. The compositions of the invention may be manufactured by processes well known in the art, for example, by means of conventional mixing, dissolving, granulating, emulsifying, encapsulating, entrapping or lyophilizing processes.

The polyalkylene glycols useful herein may be characterized by way of both their generalized preparation route and certain common aspects of their structures. Their preparation route generally involves the reaction of an alcohol and a feed that includes both butylene oxide and propylene oxide. A wide ratio of proportions of the feed oxides may be employed, such that the butylene oxide to propylene oxide ratio may range from 4:1 to 1:4, preferably from 3:1 to 1:3, and more preferably from 3:1 to 1:1, by weight. In some non-limiting embodiments a random distribution of the oxide units is preferred, while in other embodiments a block structure may be created by controlling the feed such that the oxides are fed separately and/or alternated.

In certain preferred embodiments, polyalkylene glycols useful in the invention may be prepared by the reaction of at least butylene oxide, propylene oxide, and a selected alcohol, resulting in a polyalkylene glycol compound of Formula I:

RO-(PO)ₙ-(BO)ₘ-H (I)

wherein PO is propyleneoxy, BO is butyleneoxy, R is a linear or branched C₈-C₂₀ alkyl, n has an average value of from 5 to 20, and m has an average value of from 4 to 16. In certain preferred embodiments, the butylene oxide structural unit is 1,2-butylene oxide, and is a polyalkylene glycol of Formula II:

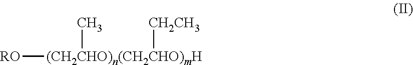

wherein R, n, and m have the same definitions as in Formula I. In certain preferred embodiments R is a linear or branched C₈-C₁₂ alkyl, and more preferably a C₁₂ alkyl. In some embodiments, a mixture of specified alcohol initiators may be selected. The alcohol initiator may be obtained from either petrochemical or renewable resources, and is in general a C₈-C₂₀ alcohol which may be linear or branched in nature, preferably a C₈-C₁₂ alcohol, and more preferably dodecanol. As used herein, designations beginning with “C,” including but not limited to C₈, C₁₀, C₁₂, and C₂₀, refer to the total number of carbon atoms in a given molecule, regardless of the configuration of these atoms. Hyphenated expressions including such carbon number designations, such as C₈-C₁₂, refer to a group of possible selections of molecules, each selection having a carbon number falling within the given numerical range.

The reaction may be catalyzed by either an acidic or basic catalyst. In certain non-limiting embodiments, the catalyst is an alkali base, such as potassium hydroxide, sodium hydroxide, or sodium carbonate, and the process is an anionic polymerization. The result is a polyether structure having a relatively narrower molecular weight distribution, that is, a relatively low polydispersity index, than may be obtained when the polymerization proceeds cationically. However, in alternative and non-limiting embodiments, cationic polymerization may be performed. The polymer chain length will also depend upon the ratio of the reactants, but in certain non-limiting embodiments the number average molecular weight (Mn) may vary from 500 to 5,000, and in certain other non-limiting embodiments may vary from 500 to 2,500.

In an alternative characterization, the polyalkylene glycols useful in the present invention may be characterized as butylene oxide/propylene oxide-extended copolymers, based on primary hydroxyl group-containing initiators and having a carbon to oxygen ratio of at least 3:1, and in certain embodiments, from 3:1 to 6:1.

A person of ordinary skill in the art can readily determine the effective amount of the polyalkylene glycol of the present invention that should be used in a particular composition in order to provide the desired benefits described herein (e.g., treatment of dry skin and/or inhibition of trans-epidermal water loss) via a combination of general knowledge of the applicable field as well as routine experimentation where needed. By way of non-limiting example, the amount of the polyalkylene glycol in the composition of the invention may be in the range of from 0.01 to 50 weight percent, preferably from 1 to 30 weight percent, and more preferably from 2 to 10 weight percent, based on the total weight of the composition.

Compositions of the invention also include a dermatologically acceptable carrier. Such material is typically characterized as a carrier or a diluent that does not cause significant irritation to the skin and does not negate the activity and properties of active agent(s) in the composition. Examples of dermatologically acceptable carriers that are useful in the invention include, without limitation, water, such as deionized or distilled water, emulsions, such as oil-in-water or water-in-oil emulsions, alcohols, such as ethanol, isopropanol or the like, glycols, such as propylene glycol, glycerin or the like, creams, aqueous solutions, oils, ointments, pastes, gels, lotions, milks, foams, suspensions, powders, or mixtures thereof. In some embodiments, the composition contains from about 99.99 to about 50 percent by weight of the dermatologically acceptable carrier, based on the total weight of the composition.

The personal care composition of the invention may also include, for instance, a thickener, additional emollients, an emulsifier, a humectant, a surfactant, a suspending agent, a film forming agent, a lower monoalcoholic polyol, a high boiling point solvent, a propellant, a mineral oil, silicon feel modifiers, or mixtures thereof. The amount of optional ingredients effective for achieving the desired property provided by such ingredients can be readily determined by one skilled in the art.

In certain embodiments, the personal care compositions of the present invention further comprise a personal care active selected from skin care actives, nail care actives, or hair care actives. Actives include sunscreens, skin colorants, drug substances (such as anti-inflammatory agents, antibiotics, topical anesthetics, antimycotics, keratolytics, and the like), skin protectants, conditioners, humectants, and ultraviolet radiation absorbers.

Other additives may be included in the compositions of the invention such as, but not limited to, abrasives, absorbents, aesthetic components such as fragrances, pigments, colorings/colorants, essential oils, skin sensates, astringents (e.g., clove oil, menthol, camphor, eucalyptus oil, eugenol, menthyl lactate, witch hazel distillate), preservatives, anti-caking agents, a foam building agent, antifoaming agents, antimicrobial agents (e.g., iodopropyl butylcarbamate), antioxidants, binders, biological additives, buffering agents, bulking agents, chelating agents, chemical additives, colorants, cosmetic astringents, cosmetic biocides, denaturants, drug astringents, external analgesics, film formers or materials, e.g., polymers, for aiding the film-forming properties and substantivity of the composition (e.g., copolymer of eicosene and vinyl pyrrolidone), opacifying agents, pH adjusters, propellants, reducing agents, sequestrants, skin bleaching and lightening agents (e.g., hydroquinone, kojic acid, ascorbic acid, magnesium ascorbyl phosphate, ascorbyl glucosamine), skin-conditioning agents (e.g., humectants, including miscellaneous and occlusive), skin soothing and/or healing agents (e.g., panthenol and derivatives (e.g., ethyl panthenol), aloe vera, pantothenic acid and its derivatives, allantoin, bisabolol, and dipotassium glycyrrhizinate), skin treating agents, and vitamins (e.g., Vitamin C) and derivatives thereof. The amount of option ingredients effective for achieving the desired property provided by such ingredients can be readily determined by one skilled in the art.

As noted above, personal care compositions of the present invention are highly effective as emollients. They exhibit emollient attributes on par with, if not better than previously known emollients for personal care applications, without the disadvantage of high comedogenic potential or environmental and health related issues. Accordingly, the personal care compositions of the present invention are useful for the treatment and protection of skin, including, for example, moisturization of the skin, prevention and treatment of dry skin, protection of sensitive skin, improvement of skin tone and texture, masking imperfections, and inhibition of trans-epidermal water loss. Thus, in one aspect the present invention provides that the personal care compositions may be used in a method for treating skin. The compositions may also be used in a method for inhibiting trans-epidermal water loss in skin.

In practicing the methods of the invention, the personal care compositions are generally administered topically by applying or spreading the compositions onto the skin. A person of ordinary skill in the art can readily determine the frequency with which the compositions should be applied. The frequency may depend, for example, on the level of humidity that an individual is likely to encounter in a given day and/or the sensitivity of the individual to low humidity. By way of non-limiting example, administration on a frequency of at least once per day may be desirable.

Some embodiments of the invention will now be described in detail in the following Examples.

### EXAMPLES

### Example 1

### Preparation of Exemplary Personal Care Formulations

Exemplary personal care compositions of the present invention contain the components recited in Table 1.

TABLE 1
Exemplary Personal Care Formulations

| | | | |
|---|---|---|---|
| Trade Name | INCI Name | E1 (w/w %) | E2 (w/w %) |

Phase I

| | | | |
|---|---|---|---|
| DI Water | Water | 90.67 | 90.67 |
| Acritamer 980 | Carbomer | 0.15 | 0.15 |
| Glycerin | Glycerin | 1.00 | 1.00 |

Phase II

| | | | |
|---|---|---|---|
| Procol CS-20-D | Cetearyl Alcohol, | 1.50 | 1.50 |
| | Ceteareth 20 | | |
| RITA GMS | Glyceryl Stearate | 1.00 | 1.00 |
| UCON OSP- | | 5.00 | — |
| 150 | | | |
| UCON OSP- | | — | 5.00 |
| 220 | | | |
| Petrolatum | Petrolatum | — | — |

Phase III

| | | | |
|---|---|---|---|
| Neolone PE | Phenoxyethanol, | 0.50 | 0.50 |
| | Methylisothiazolinone | | |

Phase IV

| | | | |
|---|---|---|---|
| NaOH 20% | Sodium Hydroxide | 0.18 | 0.18 |
| Solution | | | |
| Total | | 100.00 | 100.00 |

For Phase I, Carbomer was dispersed into water mixing at variable speeds of 300-500 rpm until all hydrates were without visible lumps and particles, at which point glycerin was added and heated to 70-75° C. while stirring continuously. Phase II was mixed separately and heated to 70-75° C. until uniform. Phase II was then added to Phase I while mixing at variable speeds of 200-400 rpm for 3-5 minutes. The resulting mixture was then cooled and Phase IV was added into the batch while mixing at variable speeds of 200-500 rpm. Once the temperature reached below 45° C., Phase III was added into the batch while mixing at variables speeds of 100-300 rpm until cooled to room temperature.

### Example 2

### Preparation of Comparative Personal Care Formulations

Comparative personal care compositions contain the components recited in Table 2.

TABLE 2
Comparative Personal Care Formulations

| | | | | | |
|---|---|---|---|---|---|
| Trade Name | INCI Name | C1 (w/w %) | C2 (w/w %) | C3 (w/w %) | C4 (w/w %) |

Phase I

| | | | | | |
|---|---|---|---|---|---|
| DI Water | Water | 90.67 | 90.67 | 90.67 | 90.67 |
| Acritamer 980 | Carbomer | 0.15 | 0.15 | 0.15 | 0.15 |
| Glycerin | Glycerin | 1.00 | 1.00 | 1.00 | 1.00 |

Phase II

| | | | | | |
|---|---|---|---|---|---|
| Procol CS-20-D | Cetearyl Alcohol, | 1.50 | 1.50 | 1.50 | 1.50 |
| | Ceteareth 20 | | | | |
| RITA GMS | Glyceryl Stearate | 1.00 | 1.00 | 1.00 | 1.00 |
| UCON Fluid | PPG-14 Butyl | 5.00 | — | — | — |
| AP | Ether | | | | |
| Petrolatum | Petrolatum | — | 5.00 | — | — |
| HydroBrite 380 | Mineral Oil | — | — | 5.00 | — |
| PO | | | | | |
| DC 200, 100 cst | Dimethicone | — | — | — | 5.00 |

Phase III

| | | | | | |
|---|---|---|---|---|---|
| Neolone PE | Phenoxyethanol, | 0.50 | 0.50 | 0.50 | 0.50 |
| | Methylisothiazolinone | | | | |

Phase IV

| | | | | | |
|---|---|---|---|---|---|
| NaOH 20% | Sodium | 0.18 | 0.18 | 0.18 | 0.18 |
| Solution | Hydroxide | | | | |
| Total | | 100.00 | 100.00 | 100.00 | 100.00 |

For Phase I, Carbomer was dispersed into water mixing at variable speeds of 300-500 rpm until all hydrates were without visible lumps and particles, at which point glycerin was added and heated to 70-75° C. while stirring continuously. Phase II was mixed separately and heated to 70-75° C. until uniform. Phase II was then added to Phase I while mixing at variable speeds of 200-400 rpm for 3-5 minutes. The resulting mixture was then cooled and Phase IV was added into the batch while mixing at variable speeds of 200-500 rpm. Once the temperature reached below 45° C., Phase III was added into the batch while mixing at variables speeds of 100-300 rpm until cooled to room temperature.

### Example 3

### Sensory Feel Panel Study—Oiliness

The formulations as prepared in Examples 1 and 2 above were evaluated by a panel of ten persons for the aesthetic attribute of oiliness after application to the skin. A performance ranking of 1 (indicating more oily feeling) to 5 (indicating less oily feeling) was assigned by each panelist to each of the formulations based upon the perceived sensorial feel during application. The average ranking assigned by the panelists is presented in Table 3.

TABLE 3
Sensory Feel Panel Study Rankings - Oiliness

| Formulation | Emollient | Oiliness Ranking |
|---|---|---|
| E1 (inventive) | UCON OSP-150 | 3.3 |
| E2 (inventive) | UCON OSP-220 | 3.6 |
| C1 (comparative) | UCON Fluid AP | 2.9 |
| C2 (comparative) | Petrolatum | 3.6 |
| C3 (comparative) | Mineral Oil | 3.5 |
| C4 (comparative) | Dimethicone | 2.8 |

The above results demonstrate that the inventive skin care formulations compared favorably with the benchmark comparative formulations containing petrolatum and mineral oil when evaluated for oiliness.

### Example 4

### Sensory Feel Panel Study—Whiteness

The formulations as prepared in Examples 1 and 2 above were evaluated by a panel of ten persons for the aesthetic attribute of whiteness after application to the skin. A performance ranking of 1 (indicating more whiteness) to 5 (indicating less whiteness) was assigned by each panelist to each of the formulations based upon the perceived whiteness after application. The average ranking assigned by the panelists is presented in Table 4.

TABLE 4
Sensory Feel Panel Study Rankings - Whiteness

| Formulation | Emollient | Oiliness Ranking |
|---|---|---|
| E1 (inventive) | UCON OSP-150 | 3.9 |
| E2 (inventive) | UCON OSP-220 | 4.4 |
| C1 (comparative) | UCON Fluid AP | 4.4 |
| C2 (comparative) | Petrolatum | 3.5 |
| C3 (comparative) | Mineral Oil | 3.3 |
| C4 (comparative) | Dimethicone | 4.8 |

The above results demonstrate that the inventive skin care formulations were perceived more favorably, i.e., imparted less whiteness, than the benchmark comparative formulations containing petrolatum and mineral oil, and compared favorably with dimethicone.

### Example 5

### Sensory Feel Panel Study—Absorption

The formulations as prepared in Examples 1 and 2 above were evaluated by a panel of ten persons for the aesthetic attribute of absorption after application to the skin. A performance ranking of 1 (indicating less absorption) to 5 (indicating more absorption) was assigned by each panelist to each of the formulations based upon the perceived absorption after application. The average ranking assigned by the panelists is presented in Table 5.

TABLE 5
Sensory Feel Panel Study Rankings - Absorption

| Formulation | Emollient | Oiliness Ranking |
|---|---|---|
| E1 (inventive) | UCON OSP-150 | 3.5 |
| E2 (inventive) | UCON OSP-220 | 3.7 |
| C1 (comparative) | UCON Fluid AP | 2.8 |
| C2 (comparative) | Petrolatum | 3.0 |
| C3 (comparative) | Mineral Oil | 2.6 |
| C4 (comparative) | Dimethicone | 3.7 |

The above results demonstrate that the inventive skin care formulations compared favorably with the benchmark comparative formulations when evaluated for absorption into the skin.

### Example 6

### Sensory Feel Panel Study—Residual Smoothness

The formulations as prepared in Examples 1 and 2 above were evaluated by a panel of ten persons for the aesthetic attribute of residual smoothness after application to the skin. A performance ranking of 1 (indicating less residual smoothness) to 5 (indicating more residual smoothness) was assigned by each panelist to each of the formulations based upon the perceived absorption after application. The average ranking assigned by the panelists is presented in Table 6.

TABLE 6
Sensory Feel Panel Study Rankings - Residual Smoothness

| Formulation | Emollient | Oiliness Ranking |
|---|---|---|
| E1 (inventive) | UCON OSP-150 | 3.4 |
| E2 (inventive) | UCON OSP-220 | 3.7 |
| C1 (comparative) | UCON Fluid AP | 3.7 |
| C2 (comparative) | Petrolatum | 3.2 |
| C3 (comparative) | Mineral Oil | 3.3 |
| C4 (comparative) | Dimethicone | 3.9 |

The above results demonstrate that the inventive skin care formulations compared favorably with the benchmark comparative formulations when evaluated for residual smoothness of the skin.

### Example 7

### Skin Hydration Panel Study

The formulations as prepared in Examples 1 and 2 above were evaluated by a panel of ten persons for moisturizing performance. The percent change in water content in the epidermal layer was evaluated one hour after application of the formulation. The higher percent change, the better the moisturizing performance. The percent change for each formulation is presented in Table 7.

TABLE 7
Skin Hydration Panel Study

| Formulation | Emollient | % Hydration Change |
|---|---|---|
| E1 (inventive) | UCON OSP-150 | 26.4 |
| E2 (inventive) | UCON OSP-220 | 25.3 |
| C1 (comparative) | UCON Fluid AP | 22.1 |
| C2 (comparative) | Petrolatum | 30.5 |
| C3 (comparative) | Mineral Oil | 26.2 |

The above results demonstrate that the inventive formulations can provide equivalent skin hydration and moisturization benefits in oil-free formulations as compared with mineral oil and petrolatum.

### Example 8

### Trans-Epidermal Water Loss Study

The formulations as prepared in Examples 1 and 2 above were evaluated by a panel of ten persons for performance as a skin barrier to prevent water loss in skin. The percent change in skin water content in the epidermal layer was evaluated one hour after application of the formulation. The higher percent change, the better the performance as a skin barrier. The percent change for each formulation is presented in Table 8.

TABLE 8
Trans-Epidermal Water Loss Panel Study

| Formulation | Emollient | % Hydration Change |
|---|---|---|
| E1 (inventive) | UCON OSP-150 | 26.4 |
| E2 (inventive) | UCON OSP-220 | 25.3 |
| C1 (comparative) | UCON Fluid AP | 22.1 |
| C2 (comparative) | Petrolatum | 30.5 |
| C3 (comparative) | Mineral Oil | 26.2 |

The above results demonstrate that the inventive formulations can provide equivalent skin barrier performance in oil-free formulations as compared with mineral oil and petrolatum.

What is claimed is:

1. A personal care composition comprising:
(a) an oil-soluble polyalkylene glycol, wherein the polyalkylene glycol comprises units derived from propylene oxide and units derived from butylene oxide; and
(b) a dermatologically acceptable carrier.

2. The personal care composition of claim 1, wherein the polyalkylene glycol is a compound of Formula I:
RO-(PO)ₙ-(BO)ₘ-H (I)
wherein PO is propyleneoxy, BO is butyleneoxy, R is a linear or branched C₈-C₂₀ alkyl, n has an average value of from 5 to 20, and m has an average value of from 4 to 16.

3. The personal care composition of claim 2, wherein R is a linear or branched C₈-C₁₂ alkyl.

4. The personal care composition of claim 2, wherein R is a linear or branched Cu alkyl.

5. The personal care composition of claim 1, wherein the polyalkylene glycol has a number average molecular weight of from 500 to 5,000.

6. The personal care composition of claim 1, wherein the weight ratio of butyleneoxy units to propyleneoxy units ranges from 4:1 to 1:4.

7. The personal care composition of claim 1, wherein the polyalkylene glycol is present in an amount of from 0.01 wt % to 50 wt %.

8. The personal care composition of claim 1, further comprising a skin care active.

9. A method of treating skin comprising topically administering to the skin the composition of claim 1.

10. A method for inhibiting trans-epidermal water loss in skin comprising topically administering to the skin the composition of claim 1.
